(19) United States
(12) Patent Application Publication
(10) Pub. No.: US 2022/0012888 A1
(43) Pub. Date: Jan. 13, 2022

(54) METHODS AND SYSTEM FOR AUTONOMOUS VOLUMETRIC DENTAL IMAGE SEGMENTATION

(71) Applicant: CARESTREAM DENTAL LLC, Atlanta, GA (US)

(72) Inventors: Shoupu CHEN, Rochester, NY (US); Wei YE, Shanghai (CN)

(21) Appl. No.: 17/293,849

(22) PCT Filed: Nov. 14, 2019
(86) PCT No.: PCT/US19/61374

§ 371 Date: May 13, 2021

Related U.S. Application Data
(60) Provisional application No. 62/767,083, filed on Nov. 14, 2018.

(51) Int. Cl. G06T 7/11; G06T 7/12; G06T 7/00; G06T 5/30; A61B 6/14; A61C 7/00; G16H 30/40; G16H 50/20; G16H 30/20

(52) U.S. Cl. CPC G06T 7/11; G06T 7/12; G06T 7/0012; G06T 5/30; A61B 6/14; A61C 7/002; G06T 2207/20161; G16H 50/20; G16H 30/20; G06T 2207/10081; G06T 2207/30036; G06T 2200/04; G06T 2207/20108; G16H 30/40

(57) ABSTRACT

The present disclosure describes a system and methods for autonomous segmentation of volumetric dental images, such as those produced by an imaging system, The methods, implemented by the system, acquire a volume image of a patient and extract a volume of interest comprising patient dentition from the acquired volume image. A first plane is extended through maxillary portions of the patients jaw and a second plane through mandibular portions of the patients jaw. A maxillary sub-volume is generated from the volume of interest according to the first plane and a mandibular sub-volume from the volume of interest according to the second plane. Maximum intensity projection images are formed for each sub-volume and teeth are delineated from these images. Teeth are segmented within each sub-volume according to the tooth delineation for their respective sub-volume.

### FIELD OF THE INVENTION

The present invention relates generally to volume image processing in x-ray computed tomography and, in particular, to image segmentation of a three-dimensional (“3D”) volume from digital Cone Beam Computed Tomography (“CBCT”).

### BACKGROUND

Imaging and image processing for computer-aided diagnosis and improved patient care are areas of interest to dental practitioners. Among areas of particular interest for computer-aided diagnosis, treatment assessment, and surgery is image segmentation, particularly, for tooth regions.

A three-dimensional or volume x-ray image can be of significant value for diagnosis and treatment of teeth and supporting structures. A volume x-ray image for this purpose is formed by combining image data from two or more individual two-dimensional (“2D”) projection images, obtained within a short time of each other and with a well-defined angular and positional geometry between each projection image and the subject tooth and between each projection image and the other projection images. Cone-Beam Computed Tomography (“CBCT”) is one established method for obtaining a volume image of dental structures from multiple projection images. In CBCT imaging, an image detector and an x-ray source orbit a subject and obtain a series of x-ray projection images at small angular increments. The information obtained is then used to synthesize a volume image that faithfully represents the imaged subject to within the available resolution of the system, so that the volume image that is formed can then be viewed from any number of angles. Commercially available CBCT apparatuses for dental applications include the CS 8100 3D System from Carestream Dental LLC of Atlanta, Ga.

For intraoral CBCT imaging, it is often useful to segment the maxilla and mandible so that upper and lower jaw features can be viewed and manipulated separately. The capability for accurate segmentation of maxilla and mandible has particular advantages for assessing how these structures work together.

Various approaches have been proposed to address tooth segmentation. For example, one researcher has described a method for automating postmortem identification of teeth for deceased individuals based on dental characteristics. Other researchers have described a method of dealing with problems of 3D tissue reconstruction in stomatology. In this method, 3D geometry models of teeth and jaw bones were created based on input computed tomography (“CT”) image data. Still other researchers have proposed a fast, automatic method for the segmentation and visualization of teeth in multi-slice CT-scan data of the patient's head. The method uses a sequence of processing steps. The mandible and maxilla are separated using maximum intensity projection (“MIP”) in the y-direction and a step-like region separation algorithm. The dental region is separated using maximum intensity projection in the z-direction, thresholding, and cropping. The teeth are segmented using a region growing algorithm. Results are visualized using iso-surface extraction and surface and volume rendering. Additionally, other researchers have disclosed a method to construct and visualize an individual tooth model from CT image sequences for dental diagnosis and treatment.

Yet other methods have been proposed that, for example, require the viewer to estimate the contour of each tooth in order to allow more efficient tooth segmentation. This estimation, however, proves to be challenging and the overall method achieves results that can often be unsatisfactory. Methods have also been proposed that require zero overlap between upper and lower teeth, which proves to be a significant constraint. Still other methods require conversion of the 3D image to a surface mesh, with often disappointing results.

Thus, although some advances have been made, achieving error-free segmentation processing continues to be a challenge. Over-segmentation, with detection of false positives, continues to be a chronic difficulty with volume images of patient dentition, particularly where teeth are within very close proximity of each other. There is a desire to correctly differentiate foreground from background areas in a volume image.

Therefore, there is a need in the industry for a system and methods for autonomous volumetric dental image segmentation that resolves these and other problems, difficulties, and shortcomings of present systems and methods of segmenting a volumetric dental image.

### SUMMARY OF THE INVENTION

Broadly described, the present invention comprises a system and methods for autonomous segmentation of volumetric dental images that are defined by the appended claims. Such volumetric dental images include, but are not limited to, cone beam computed tomography volumetric dental images, computed tomography volumetric dental images, intraoral volumetric dental images, and volumetric dental images produced by other systems or technologies available now or in the future. According to an example embodiment of the present disclosure, there is provided a method comprising the steps of: acquiring a volume image of a patient; identifying a first plane extending through maxillary portions of the patient's jaw and a second plane extending through mandibular portions of the patient's jaw; and generating a maxillary sub-volume from the volume of interest according to the first plane and a mandibular sub-volume from the volume of interest according to the second plane.

These and other inventive methods, systems, aspects or features of the present invention will become apparent from reviewing and considering the text and drawings of the present disclosure.

### BRIEF DESCRIPTION OF THE DRAWINGS

FIG. 1 is a schematic diagram showing an imaging apparatus for CBCT imaging of the head.

FIG. 2 is a schematic diagram that shows how CBCT imaging acquires multiple radiographic projection images for reconstruction of the 3D volume image.

FIG. 3 is a flowchart that shows a method used for tooth segmentation for a dental Cone Beam Computed Tomography (CBCT) volume according to an embodiment of the present disclosure.

FIGS. 4A and 4B show examples of a visualization tool utility that generates and displays a visualization tool window for plane placement in a plane positioning step of FIG. 3.

FIG. 5 is a flowchart that shows a method for identifying and segmenting or extracting a volume of interest (VOI).

FIGS. 6A-6F are schematic diagrams that show a sequence for separation of the VOI to form a maxilla sub-volume and a mandible sub-volume.

FIG. 6G shows plane placement of FIG. 3A with 3D visualization.

FIG. 6H shows the 3D counterpart of FIG. 6F for a mandible sub-volume.

FIG. 6I shows the 3D counterpart of FIG. 6F for a maxilla sub-volume.

FIG. 7 is a flowchart showing a method for generating maximum intensity projection (MIP) images from the separated sub-volumes.

FIGS. 8A-8E are images corresponding to steps of the method of FIG. 7.

FIGS. 9A, 9B, and 9C show initial stages of the tooth delineation within the MIP images of the respective sub-volumes.

FIGS. 10A and 10B show processing to identify a separating line between adjacent teeth.

FIG. 11 is a graph showing measurements that identify tooth separation positions along center line 56 in FIG. 9C.

FIG. 12 shows an exemplary outline view of tooth separation structure in the processed MIP for a jaw.

FIG. 13A shows an initial separation of teeth in an MIP image.

FIG. 13B shows improved separation of teeth in an MIP image after subsequent processing.

FIG. 14 shows an example of the initial CBCT slice segmentation corresponding to a positioned plane.

FIGS. 15 and 16 show, from different views, exemplary 3D volume rendered segmented teeth.

FIG. 17 shows principal axes for a patient's teeth according to an example embodiment of the present disclosure.

FIG. 18A shows a segmentation error with contour concavity.

FIG. 18B shows contour concavity where segmentation is correct.

FIG. 19 shows an alternate example of a segmentation error due to ambiguities in bone/root interpretation.

### DETAILED DESCRIPTION OF EXAMPLE EMBODIMENTS

In the following detailed description of example embodiments of the present invention, reference is made to the drawings in which the same reference numerals are assigned to identical elements or steps in successive figures. It should be noted that these figures are provided to illustrate overall functions and relationships according to embodiments of the present invention and are not provided with intent to represent actual size or scale.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the terms “viewer”, “operator”, and “user” are considered to be equivalent and refer to the viewing practitioner, technician, or other person who views and manipulates an image, such as a dental image, on a display monitor. An “operator instruction” or “viewer instruction” is obtained from explicit commands entered by the viewer, such as by clicking a button on a camera or by using a computer mouse or by touch screen or keyboard entry.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present invention, the descriptive terms “object of interest” or “feature of interest” generally indicate an object such as a tooth or other object in the mouth.

The term “set”, as used herein, refers to a non-empty set, as the concept of a collection of elements or members of a set is widely understood in elementary mathematics. The term “subset”, unless otherwise explicitly stated, is generally used herein to refer to a non-empty proper subset, that is, to a subset of the larger set, having one or more members. For a set “S”, a subset may comprise the complete set “S”. A “proper subset” of set “S”, however, is strictly contained in set “S” and excludes at least one member of set “S”.

In the context of the present disclosure, the terms “pixel” and “voxel” may be used interchangeably to describe an individual digital image data element, that is, a single value representing a measured image signal intensity. Conventionally, an individual digital image data element is referred to as a voxel for 3-dimensional volume images and a pixel for 2-dimensional images. Volume images, such as those from CT or CBCT apparatus, are formed by obtaining multiple 2D images of pixels, taken at different relative angles, then combining the image data to form corresponding 3D voxels. For the purposes of the description herein, the terms voxel and pixel can generally be considered equivalent, describing an image elemental datum that is capable of having a range of numerical values. Voxels and pixels have the attributes of both spatial location and image data code value.

For general description and background on CT imaging, reference is hereby made to U.S. Pat. No. 8,670,521 entitled “Method for Generating an Intraoral Volume Image” by Bothorel et al., commonly assigned.

Overview of Dental CBCT Apparatus

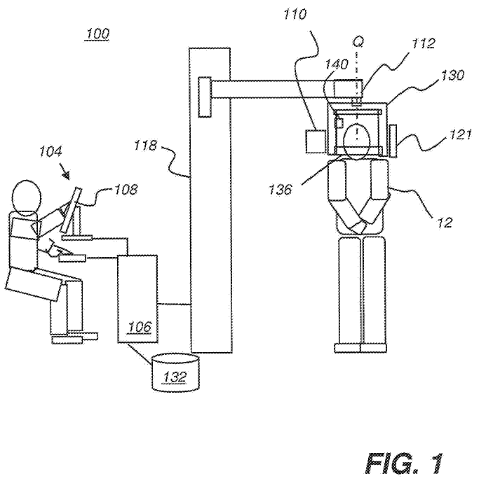

The schematic diagram of FIG. 1 shows an imaging apparatus 100 for 3D CBCT cephalometric imaging, including dental imaging. For imaging a patient 12, a succession of multiple 2D projection images is obtained and processed using imaging apparatus 100. A rotatable mount 130 is provided on a column 118, preferably adjustable in height to suit the size of patient 12. Mount 130 maintains an x-ray source 110 and a radiation sensor 121 on opposite sides of the head of patient 12 and rotates to orbit source 110 and sensor 121 in a scan pattern about the head. Mount 130 rotates about an axis Q that corresponds to a central portion of the patient's head, so that components attached to mount 130 orbit the head. Sensor 121, a digital sensor, is coupled to mount 130, opposite x-ray source 110 that emits a radiation pattern suitable for CBCT volume imaging. An optional head support 136, such as a chin rest or bite element, provides stabilization of the patient's head during image acquisition. A computer 106 has an operator interface 104 and a display 108 for accepting operator commands and for display of volume images of the orthodontia image data obtained by imaging apparatus 100. Computer 106 is in signal communication with sensor 121 for obtaining image data and provides signals for control of source 110 and, optionally, for control of a rotational actuator 112 for mount 130 components. Computer 106 is also in signal communication with a memory 132 for storing image data. An optional alignment apparatus 140 is provided to assist in proper alignment of the patient's head for the imaging process.

Volume Image Reconstruction from Multiple Projection Images

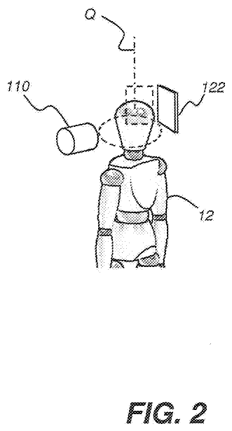

The schematic diagram of FIG. 2 shows how CBCT imaging acquires multiple radiographic projection images for reconstruction of the 3D volume image. X-ray source 110 and detector 122 revolve about patient 12 to acquire a 2D projection image at each of a number of rotational angles about axis Q. Reconstruction methods, such as filtered back projection (FBP) or other methods, apply the information from each projection image in order to generate a 3D image volume.

CBCT imaging apparatus and the imaging algorithms used to obtain 3D volume images using such systems are well known in the diagnostic imaging art and are, therefore, not described in detail in the present application. Some exemplary methods and approaches for forming 3D volume images from the source 2D images, projection images that are obtained in operation of the CBCT imaging apparatus can be found, for example, in the teachings of U.S. Pat. No. 5,999,587 entitled “Method of and System for Cone-Beam Tomography Reconstruction” to Ning et al. and of U.S. Pat. No. 5,270,926 entitled “Method and Apparatus for Reconstructing a Three-Dimensional Computerized Tomography (CT) Image of an Object from Incomplete Cone Beam Data” to Tam.

In typical applications, a computer or other type of dedicated logic processor act as control logic processor for obtaining, processing, and storing image data is part of the CBCT system, along with one or more displays for viewing image results, as shown in FIG. 1. A computer-accessible memory 132 is also provided, which may be a memory storage device used for longer term storage, such as a device using magnetic, optical, or other data storage media. In addition, the computer-accessible memory can comprise an electronic memory such as a random access memory (RAM) that is used for shorter term storage, such as employed to store a computer program having instructions for controlling one or more computers to practice the method according to methods of the present disclosure.

The subject matter of the present disclosure relates to digital image processing and computer vision technologies that process data from a digital image to recognize and thereby assign useful meaning to human-understandable objects, attributes, or conditions, and then to utilize the results obtained in further processing of the digital image.

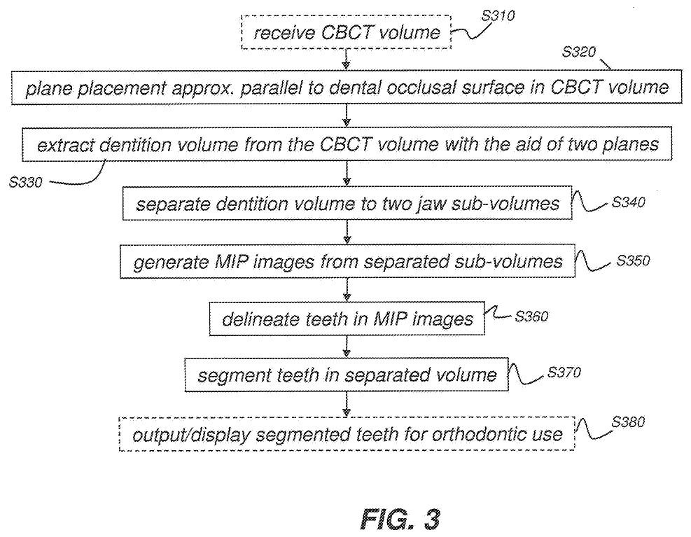

Referring to the flowchart of FIG. 3, there is shown a sequence of steps used for tooth segmentation for a dental Cone Beam Computed Tomography (CBCT) volume according to an embodiment of the present disclosure. A volume acquisition step S310 acquires a CBCT volume, prepared previously, such as using the imaging apparatus 100 shown in FIG. 1. In a plane positioning step S320, the viewer manually positions upper and lower planes on a volume rendition of the reconstructed image. Planes are positioned to intersect upper and lower crown sections, respectively. Computer 106 (FIG. 1) stores the position data for the planes and uses this information for subsequent processing steps.

Continuing with the FIG. 3 steps, a volume of interest (VOI) extraction step S330 then automatically extracts an initial full dentition VOI that contains all of the teeth, aided by the plane positioning obtained from step S320. A jaw segmentation step S340 can then be automatically executed, dividing the full VOI into a maxilla sub-volume (associated with one of the positioned planes from step S320) and a mandible sub-volume (associated with the other positioned plane).

Within each defined sub-volume from step S340 of FIG. 3, a Maximum Intensity Projections (MIP) generation step S350 then generates MIP images. A MIP image is generated from each respective sub-volume from step S340, with the normal to an MIP image aligned with the normal of the plane associated with the corresponding sub-volume, as positioned by the operator in step S320. MIPs are formed using values aligned along the normal, following practices familiar to those skilled in the imaging arts. Following MIP generation, a tooth delineation step S360 automatically delineates teeth regions in each respective set of MIP image data. This processing generates 2D tooth masks or 2D tooth contours that can be used in subsequent steps for propagation into the 3D image volume.

Using the mask or contour information obtained from step S360 of FIG. 3, a segmentation step S370 can automatically segment teeth, identifying individual teeth within each mandibular and maxillary sub-volume using the processed MIP image contours. An output step S380 then provides segmented tooth images for each tooth for display or subsequent processing.

The progression shown in FIG. 3 approaches the problem of 3D tooth segmentation in steps that use both 2D and 3D information in performing the following sequence:

(i) define the overall 3D volume that includes the dentition;

(ii) define upper and lower sub-volumes within the overall volume;

(iii) generate 2D MIP images within each respective sub-volume;

(iv) delineate teeth within the MIP images to obtain 2D mask or contour information;

(v) apply the 2D mask or contour information to 3D segmentation.

Subsequent description gives more detail on each of the processing steps outlined in the FIG. 3 method.

Plane Positioning Step S320

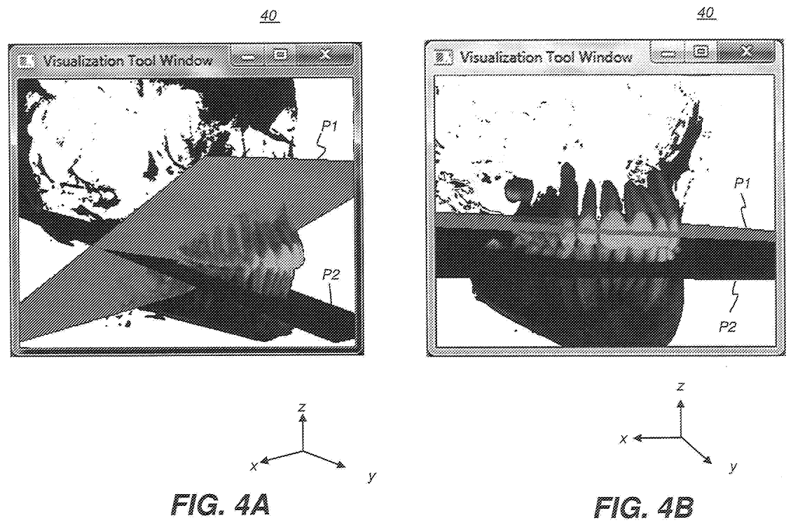

FIGS. 4A and 4B show examples of a visualization tool utility that generates and displays a visualization tool window 40 for plane placement in plane positioning step S320 of the FIG. 3 method. Planes P1 and P2 of different colors or tones are initially placed for adjustment of position against an image of the CBCT volume. As shown in FIG. 4A, the tool window 40 initially positions an upper plane P1 and a lower plane P2 for operator positioning using a cursor, touchscreen, or other screen pointing utility. Initial plane P1, P2 positioning can use default positioning used for all patients or can take advantage of image processing to approximate suitable plane placement for the particular patient.

Using conventional operator interface tools (not shown), the operator can perform various on-screen positioning tasks, including:
(i) Rotate, zoom, and translate for both planes P1 and P2 individually or for the full composite image that includes the planes P1 and P2 and the displayed volume rendition.
(ii) Specify the position of each individual plane P1, P2, such as using rotation and translation, with the volume maintained in a given position.

FIG. 4B shows an example in which planes P1 and P2 are suitably placed for subsequent processing. Planes are typically placed with plane P1 extending through the upper teeth, preferably aligned with the upper teeth crown section, and plane P2 correspondingly extended through the lower teeth, preferably aligned with the lower teeth crown section. Planes can alternately be aligned to other tooth structure. The operator can adjust and readjust plane P1, P2 position/orientation.

An exemplary guideline is to provide plane placement that helps with the following:
(i) Extracting the whole dentition section;
(ii) Guiding the separation of upper and lower dentition sections (upper and lower jaws);
(iii) Producing two MIP images each of which contains distinct teeth shapes that, in turn, facilitate generating satisfactory teeth 2D masks or teeth 2D contours that the subsequent automatic tooth segmentation process utilizes.

Approximate x, y, and z orthogonal coordinate axes are represented in each of FIGS. 4A and 4B.

According to an alternate example embodiment of the present disclosure, planes P1 and P2 are automatically positioned by the system processor. To do this, system logic can execute a series of steps such as the following:
(i) identify upper and lower tooth crowns in the CBCT volume;
(ii) estimate an occlusal plane based on the identified crowns; and
(iii) generate two planes approximately parallel to the estimated occlusal plane wherein the planes intersect their corresponding crown sections; wherein the two planes are apart from each other within a predetermined distance, such as within an exemplary distance of 15 pixels (assuming 300 microns per pixel).

VOI Extraction Step S330

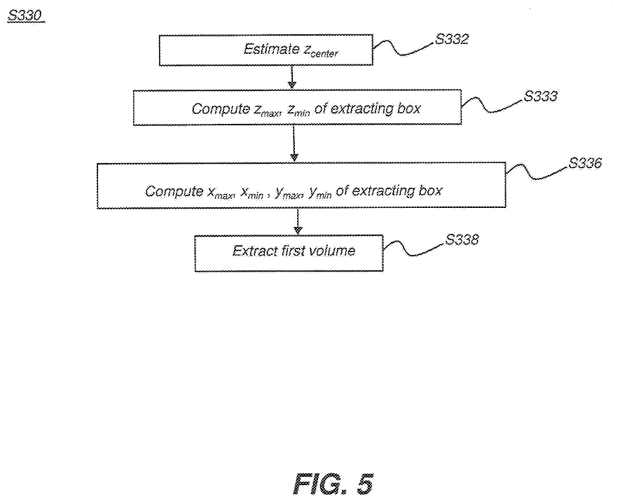

The logic flow diagram of FIG. 5 shows a processing sequence for identifying and segmenting or extracting the VOI in step S330 of the FIG. 3 sequence. The goal of this processing is to extract, from the complete CBCT volume, the full volume portion that contains all of the teeth.

Initially, the x, y, z axes shown in FIGS. 4A and 4B are determined, to be used for defining a box-shaped extracting volume from the complete CBCT volume, based on plane P1, P2 placement.

Following the sequence of FIG. 5, an estimation step S332 estimates the center height z_{center} using the average Z value of the two planes P1 and P2, with or without being weighted using the CBCT Hu (Hounsfield) values for plane intersections with the teeth.

A box computation step S333 computes zₘₐₓ and zₘᵢₙ values of the extracting box according to the estimated z_{center} value and the average tooth height, obtained from prior knowledge, such as stored values from statistical sampling or values entered for the particular patient. A computation step S336 then computes values xₘₐₓ, xₘᵢₙ, yₘₐₓ, and yₘᵢₙ that define the other two dimensions of the extracting box.

Jaw Segmentation Step S340

For orthodontic applications, the patient is asked to have the mouth closed during imaging, so that upper and lower teeth are in contact with each other. Therefore, jaw separation or segmentation is desirable for an automatic tooth segmentation system.

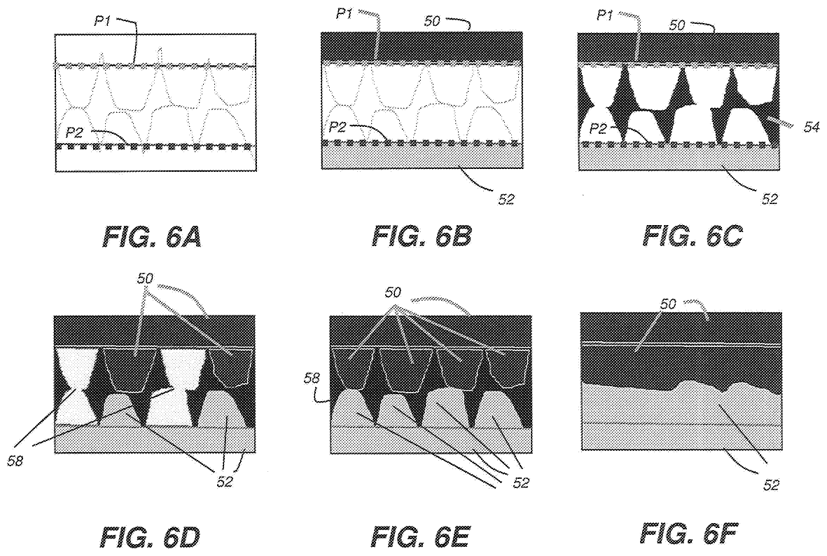

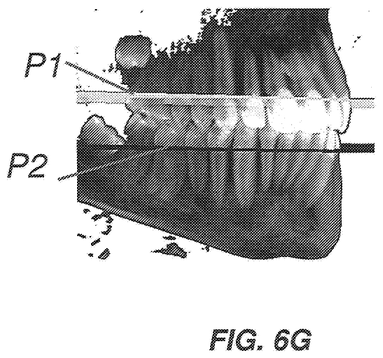

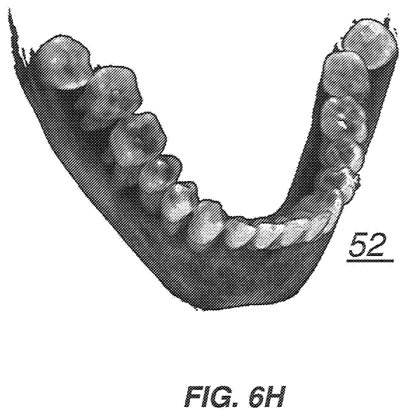

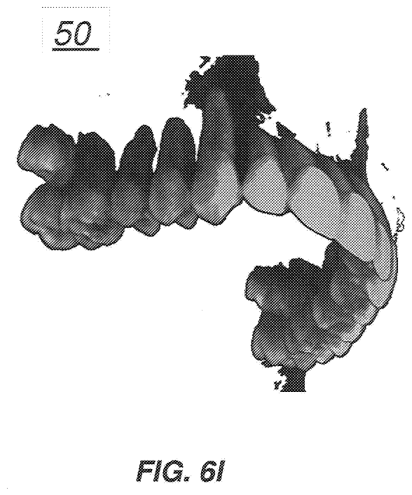

FIGS. 6A-6F are schematic diagrams that show a sequence for separation of the VOI to form a maxilla sub-volume 50 and a mandible sub-volume 52. FIG. 6A shows placement of planes P1 and P2 (indicated with dashed lines), as provided by the operator or, alternatively, by system logic, to give an initial hint to processing logic. FIG. 6B shows an initial, coarse separation for upper and lower jaw volumes 50 and 52. Extending above plane P1 is the initial maxilla sub-volume 50. Extending below plane P2 is the initial mandible sub-volume 52. The volume between planes P1 and P2 is as yet undefined.

FIG. 6C shows a classification method that is used for the volume portion that lies between planes P1 and P2. This method coarsely segments this portion of the volume into bone-like (or dentine) regions, shown in white, and non-bony regions, shown black.

FIG. 6D shows refinements to the definition of bony regions from the FIG. 6C processing. Connectivity can be analyzed using well-known image processing utilities; where a bony region connects only to the upper plane P1, that region is classified as part of maxilla sub-volume 50. Conversely, where a bony region connects only to the lower plane P2, that region is classified as part of mandible sub-volume 52. A region that was originally classed as a bony region but that fails to exhibit connection to either the upper plane P1 or lower plane P2 is reclassified as non-bony. A region 58 that appears to be connected to both planes P1 and P2 receives further analysis, as described subsequently.

FIG. 6E shows processing to separate a bony region 58 that appears to have connection with both upper and lower jaw structures. According to an example embodiment, a Random Walk method is applied for more accurate region determination. The Random Walk method, familiar to those skilled in the image processing arts, labels each pixel as part of an imaged object or background according to a suitable cost function criterion for volume segmentation.

FIG. 6F shows final segmentation results that can use, for example, a straightforward growing method that begins with a non-ambiguous bony region for completing the segmentation of maxillary and mandibular structures.

FIGS. 6G, 6H, and 6I show portions of the FIG. 6A-6F sequence as they can appear for 3D images on the graphical user interface (GUI). FIG. 6G shows the 3D counterpart of FIG. 6A, with planes P1 and P2 visualized on a 3D display of the patient's dentition. FIG. 6H shows the 3D counterpart of FIG. 6F for mandible sub-volume 52. FIG. 6I shows the 3D counterpart of FIG. 6F for maxilla sub-volume 50.

MIP Generation Step S350

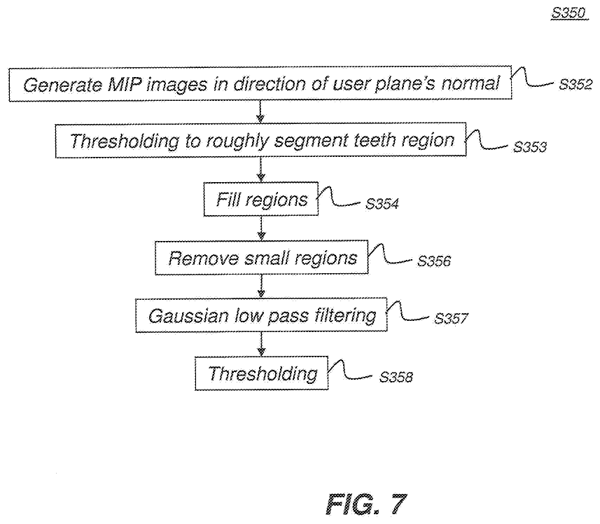

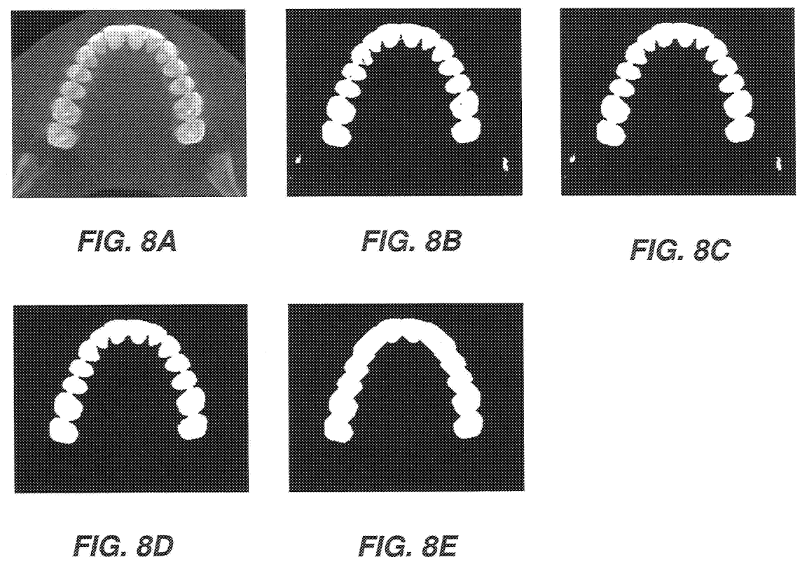

FIG. 7 is a flowchart showing a method for generating MIP images from the separated sub-volumes 50 and 52 in step S350 of FIG. 3 and preparing the MIP image for finding individual masks or contours in later steps. Stages in this method are shown in the exemplary sequence of FIGS. 8A-8E.

In an MIP formation step S352, processing forms two separate MIPs, one for maxilla sub-volume 50 and one for mandible sub-volume 52. Each respective MIP is formed using data content considered in the direction of a normal to the corresponding plane P1, P2. For each sub-volume 50, 52, this maximum intensity projection method begins, for example, at the intersection (with either plane P1 or P2) of a line that is parallel to the normal of the corresponding plane P1 or P2, assessing the intensity value of each voxel along the line, retaining the maximum (or higher) value voxel; continuing the same “assessing and retaining” projection method for all the voxel data along the same line, to the cusps of the respective teeth. This projection method repeats for all intersection points, resulting in an exemplary MIP image that contains more distinct patterns of the teeth compared to the surrounding background, as shown in FIG. 8A. A thresholding step S353 in FIG. 7 coarsely segments tooth regions in the generated MIP image, as shown in the example of FIG. 8B. A region fill step S354 then fills the segmented tooth regions to remove any holes existing inside an individual tooth region. This helps to eliminate undetermined or ambiguous areas, as shown in the example of FIG. 8C.

Continuing with the FIG. 7 steps, a small region removal step S356 removes small regions from the image that has been subject to thresholding and filling in preceding steps S353 and S354, as shown in FIG. 8D. Small, disconnected regions may be bones or noise. Following this step, provided there are no missing teeth in the original volume, only one long, curve shaped, connected tooth region remains. A filtering step S357 then performs a low-pass filtering, such as using a Gaussian low-pass filter, to process the image and obtain a smoothed image. A final thresholding step S358 then applies thresholding to obtain the final smoothed connected tooth region to remove the background, typically in the form of a curved band as shown in FIG. 8E.

Tooth Delineation Step S360

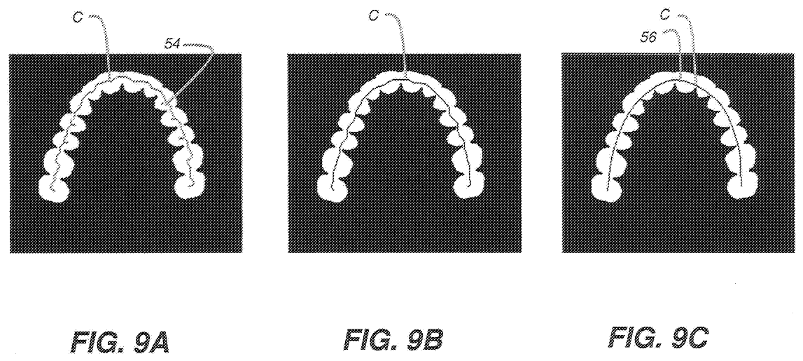

FIGS. 9A, 9B, and 9C show initial stages of the tooth delineation within the 2D MIP images of the respective sub-volumes. Tooth delineation is performed in step S360 of FIG. 3 in order to separate and identify individual teeth.

Delineation uses a smoothed medial axis or center line C as a type of geometric spline 56 for the connected tooth region for each jaw sub-volume 50, 52. In FIG. 9A, there is shown a thinning method that computes a skeletal line through centers of the smoothed connected region. FIG. 9B shows a smoothing step that removes branching features 54 to improve the center line C approximation given in FIG. 9A. In FIG. 9C, sampling along the center line C generates a number of control points for forming a smoothed spline 56 as the final center line C that spans central portions of teeth in the corresponding jaw.

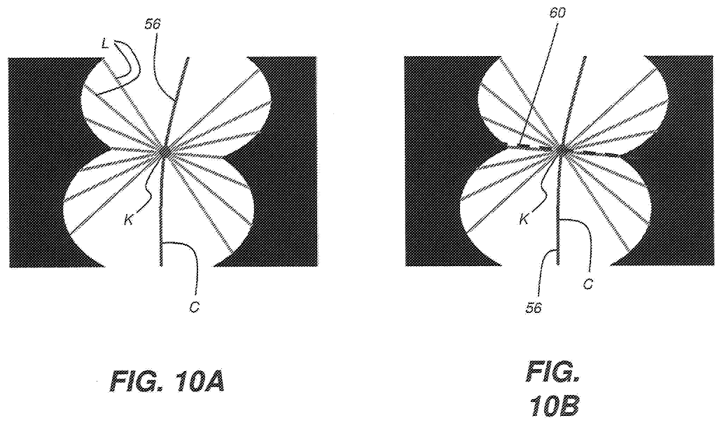

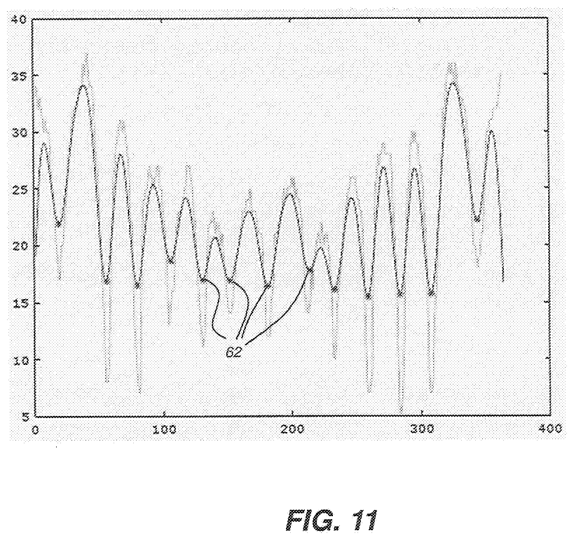

FIGS. 10A and 10B show generation of a separating line 60 that marks the space between adjacent teeth. A portion of the smoothed connected tooth region for one exemplary point K is shown in this example. From point K on the center line C, the white tooth region width is determined by extending a number of lines L at different angles through point K and measuring the distance from point K to the edge of the tooth outline along each extended line L in an attempt to identify a local minimum that indicates likely position of separating line 60. The shortest lines along the center line C, from one end to the other end, are stored as length vectors for subsequent processing. Alternately, line lengths can be measured along lines normal to the curve of spline 56.

FIG. 11 is an exemplary graph showing values of vectors of shortest length, for multiple spatial points K located along center line C. For this example, fewer than 400 spatial points along center line C are used; these points correspond to the abscissa of the graph. The ordinate for each point indicates the shortest vector length from the corresponding spatial point.

A Gaussian or other low-pass filter serves to smooth the length vector data and to reduce or eliminate spurious data and noise. The filtered length data are plotted as the oscillating bold curve in FIG. 11. A series of separating tooth interval points 62 at local minima represent approximate interdental locations for separating lines 60 in FIG. 10B, delineating the approximate location of gaps between teeth. Further processing can be provided to remove false positives.

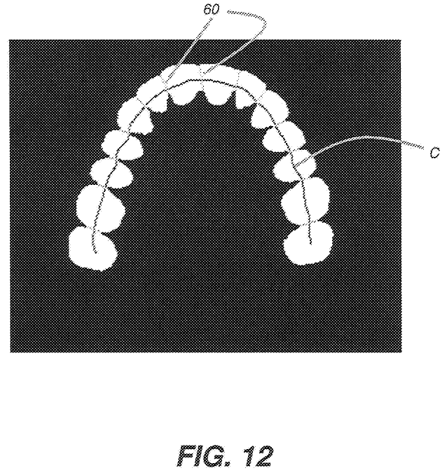

FIG. 12 shows an exemplary outline view of tooth separation structure in the processed MIP for a jaw, with separating lines 60 computed using the process described with reference to FIGS. 9A-11. The processing results shown in FIG. 12 provide a mask or “template” for coarse segmentation of tooth features in the two-dimensional MIP image that corresponds to the plane P1 or P2 position.

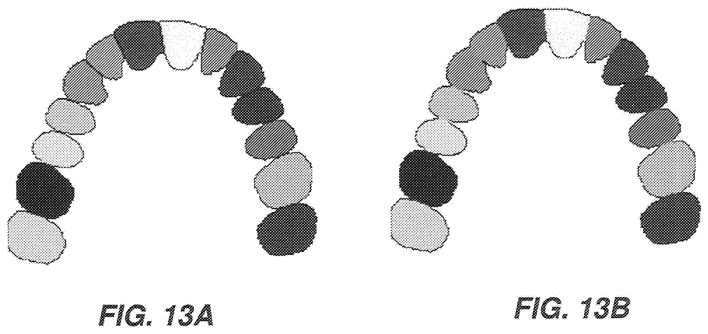

FIG. 13A shows an initial separation of teeth in a MIP image using the previously described steps S320 through S360 of FIG. 3, in which false positives can result from straight lines through separate teeth. FIG. 13B shows improved separation of teeth in a MIP image from subsequent processing using a random walk method, familiar to those skilled in the imaging arts, which greatly reduces false positives for tooth separation.

Segmentation Step S370

Segmentation step S370 of the FIG. 3 method uses the tooth contour results of MIP tooth delineation and segmentation for segmentation of the respective mandible and maxilla sub-volumes. According to an example embodiment, the sub-volume is segmented slice-by-slice. Rather than using slices defined by the CBCT system, the slice spatial orientation and angle can be determined by the plane P1, P2 positioning. The first segmented slice thus corresponds to the position of the user-placed plane P1 or P2. This segmentation processing uses the MIP segmentation results for each tooth to generate initial, coarse contours. This initial processing can then serve as input to a level set processing method, well known to those skilled in the image segmentation field, in order to more accurately segment the volume for each tooth.

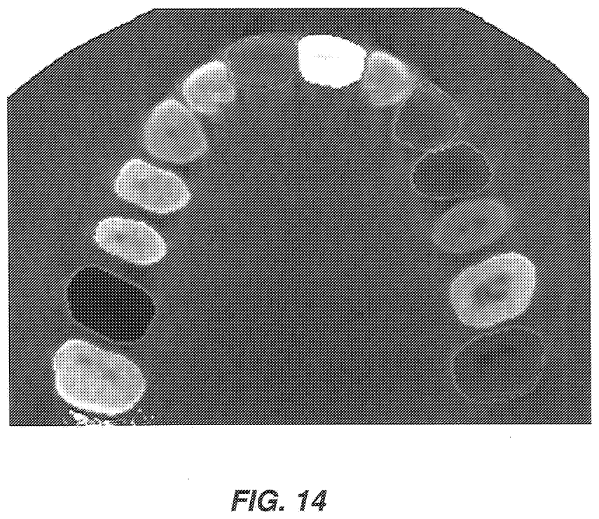

FIG. 14 shows an example of an initial slice segmentation corresponding to the positioned plane. Segmentation of the corresponding upper or lower sub-volume defined in step S340 of FIG. 3 proceeds as follows:
(i) Moving along the normal of the user placed plane, in both tooth cusp direction and tooth root direction, segment each successive slice until reaching the limit of the sub-volume;
(ii) Within each slice, use results of the previous slice to generate initial contours for each tooth;
(iii) Segment the tooth in the current slice using level set methods, or other appropriate segmentation utilities, with the aid of the initial contours.

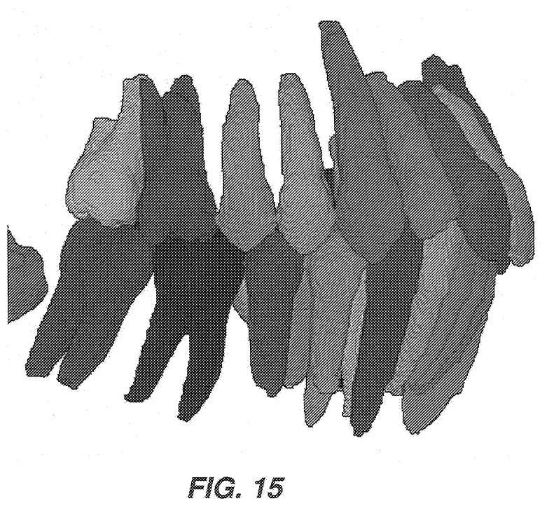

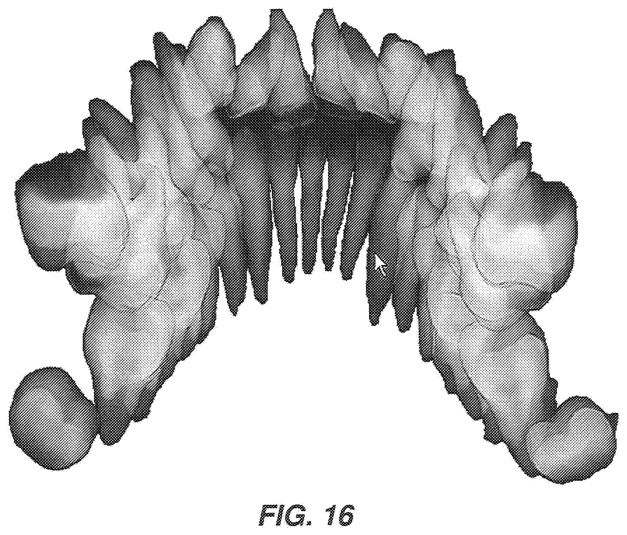

Cumulative segmentation results in all slices being grouped together to obtain 3D tooth segmentation results for the corresponding sub-volume. FIGS. 15 and 16 show, from different views, the exemplary 3D volume rendered segmented teeth.

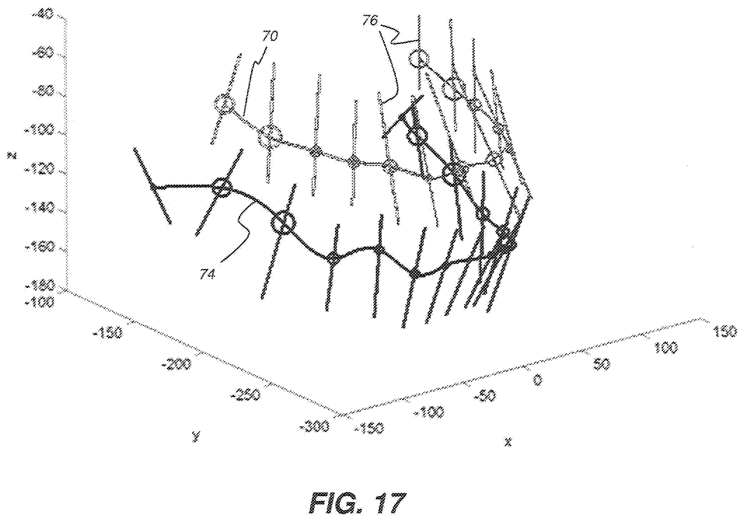

According to an example embodiment of the present disclosure, after individual teeth are segmented separately (FIG. 15), an inertia system is computed for each tooth using the (x,y,z) position of voxels of the tooth. Optionally, intensity values of the voxels can be used as weights. Usually the longest principal axis of the inertia system is chosen as a medial axis 76 of a tooth as displayed in FIG. 17. These medial axes of all the teeth can be used as cephalometric parameters in orthodontic analysis for malocclusion diagnosis or alveolar structure asymmetry diagnosis, for example. Also shown in FIG. 17 are two curves, a maxilla curve (upper) 70, and a mandibular curve (lower) 74. Each of these two curves is computed using the origins of the inertia system of the corresponding teeth.

Handling for False Positives/False Negatives

Identifying and compensating for false positives and false negatives can help to markedly improve the accuracy of segmentation step S370 of FIG. 3. The distinction can be considered as follows:
(i) For a false positive condition, there is material incorrectly added to or included with the tooth of interest. For example, a nearby region of bone may have been incorrectly incorporated into the tooth of interest in the segmentation.
(ii) For a false negative condition, there is material incorrectly omitted from the tooth of interest. For example, some portion of the tooth material may be incorrectly classified as adjacent bone.

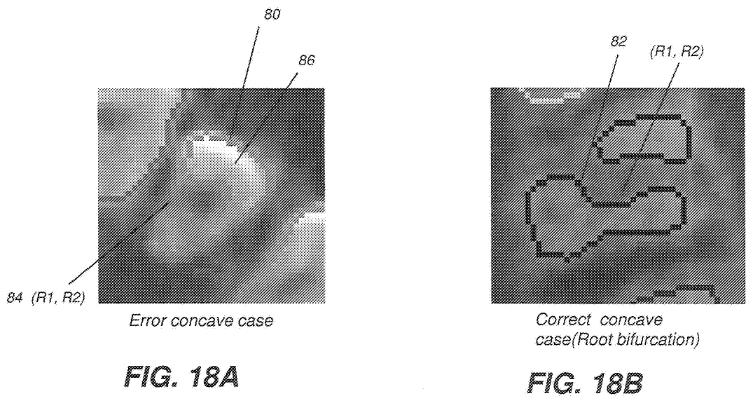

An exemplary false negative 84 in tooth segmentation is presented in the axial view of FIG. 18A. Here, due to the non-uniformity of the intensity distribution within the true tooth region (the middle tooth of interest), some portion of the actual tooth is not included in the segmentation. In the particular tooth example of FIG. 18A, it is observable that the intensity values are higher in region 86 than in region 84. The non-uniform intensity within the tooth region may result from a number of causes, including metal artifacts, photon starvation, or low X ray dose, for example.

An example embodiment of the present disclosure addresses the task of reducing the number of false negatives of the type shown in FIG. 18A by applying an approach that considers basic observations for overall tooth shape in the axial view, as follows.
(i) Convex contour. The contour 80 of a segmented tooth in an axial view should be generally a convex shape. For some types of false negative, the contour 80 of the segmented tooth exhibits a concave shape as in the example shown. Since contour concavity is irregular for axial slices through large sections of the tooth, segmentation may require some amount of correction.
(ii) Concave contour. Although convex contours most often apply for axial slices of teeth, there are situations where concave shaped contours occur in perfect tooth segmentation results. FIG. 18B shows one example wherein a contour 82 correctly shows a concave shape, correctly representing root bifurcation, showing the shapes of two connected roots of a molar.

Concavity, particularly for exposed tooth surfaces, can often suggest a segmentation error with many types of teeth. According to an example embodiment of the present disclosure, the following method steps can be executed to differentiate a “correct” concave contour from an “erroneous” concave contour:
1. Comparison step. This step compares the segmentation result of the current slice with results from the previous, adjacent slice. This comparison can include identifying a region R1, as shown in FIGS. 18A and 18B, wherein there is a significant transition of the level-set function for a number of pixels/voxels from positive (within object boundaries) to negative (outside object boundaries). The comparison step can also be performed using Sorensen-Dice coefficient metrics familiar to those skilled in the art.
2. Erosion step. This step applies an erosion operation to region R1 resulting in an eroded region R2. If region R2 is of sufficient size (for example, >20% of the region enclosed by contour 80 or contour 82), there is a high probability that contour 80 (or 82) is in a concave shape, determined automatically by the segmentation system.
3. Analysis step. This step applies statistical analysis to pixel/voxel intensity values within region R2. If the statistical analysis yields a uniform intensity distribution as in the case of FIG. 18B, concavity of contour (82) is considered to be correct, and the processing sequence for false negative detection terminates. Else, an “erroneous” concave case is found, as in FIG. 18A contour 80. If this occurs, processing responds, such as by activating the shape-prior term in the level-set segmentation algorithm in Step S370 and repeating the segmentation process for the current slice.

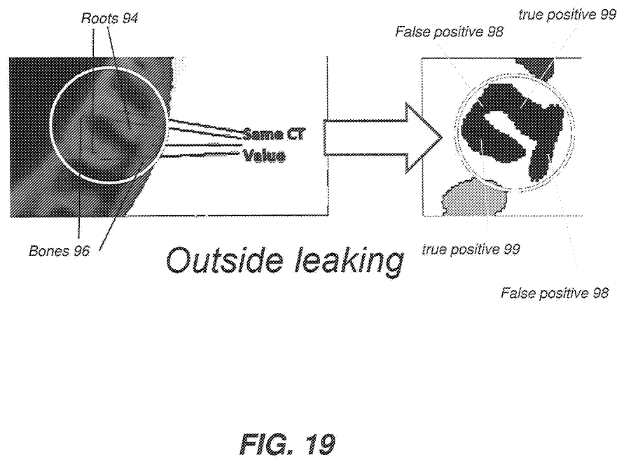

Another example of a false positive error related to ambiguous bone/root distinction is shown in FIG. 19. Here, segmentation results show a form of “leakage” outside of the root region, wherein bones 96 are mistakenly treated as roots 94 because of low contrast between different materials (roots, bones). The segmentation result is a section formed of connected pixels/voxels 92, wherein the section includes bones and roots due to the false positives; the segmentation result contains true positives 99 (roots) and false positives 98 (bones). This type of segmentation error can also be detected readily by using Sorensen-Dice coefficient metrics.

A method to correct for the exemplary false positive condition of FIG. 19 is outlined below:
1. Generate an intermediate result using a region-driven level-set segmentation.
2. Process the intermediate result by applying an edge-driven level-set segmentation on the intermediate result to yield the final, improved result.

To prevent segmentation with leaking to outside of the tooth (or roots) region, additional forces can be introduced in the level-set energy functions as follows:
(i) For region-driven level-set segmentation, as it tends to expand to neighboring teeth-of-non-interest or bones having similar intensity pixels, add a shrink-force to the level-set algorithm. This shrink-force can prevent “outside” leaking, in which false positive results spread and encroach upon true negative regions, that is, background regions (such as bones, teeth-of-non-interest).
(ii) For edge-driven level-set segmentation, as it tends to snap to strong edges and to maintain them, an expand-force can be applied. Application of this force can help to prevent “inside” leaking, in which a false negative enters true positive regions, such as tooth or root regions.

Example embodiments of the present invention provide an automated tooth segmentation system that, beginning with a reconstructed 3D volume, identifies upper and lower jaw sub-volumes, generates and processes MIP image content for each sub-volume, and applies 2D MIP segmentation results to segmentation of the complete 3D volume image.

Consistent with an example embodiment of the present invention, a computer executes a program with stored software instructions that perform on image data accessed from an electronic memory, to provide panoramic presentation and tooth segmentation in accordance with the method described. As can be appreciated by those skilled in the image processing arts, a computer program of an example embodiment of the present invention can be utilized by a suitable, general-purpose computer system, such as a personal computer or workstation. However, many other types of computer systems can be used to execute the computer program of the present invention, including networked processors. The computer program for performing the methods of the present invention may be stored in a computer readable storage medium. This medium may comprise, for example; magnetic storage media such as a magnetic disk (such as a hard drive) or magnetic tape or other portable type of magnetic disk; optical storage media such as an optical disc, optical tape, or machine readable bar code; solid state electronic storage devices such as random access memory (RAM), or read only memory (ROM); or any other physical device or medium employed to store a computer program. The computer program for performing the methods of the present invention may also be stored on computer readable storage medium that is connected to the image processor by way of the internet or other communication medium. Those skilled in the art will readily recognize that the equivalent of such a computer program product may also be constructed in hardware.

It is noted that the computer program product of the present invention may make use of various image manipulation methods and processes that are well known. It will be further understood that the computer program product example embodiment of the present invention may embody methods and processes not specifically shown or described herein that are useful for implementation. Such methods and processes may include conventional utilities that are within the ordinary skill of the image processing arts. Additional aspects of such methods and systems, and hardware and/or software for producing and otherwise processing the images or co-operating with the computer program product of the present invention, are not specifically shown or described herein and may be selected from such methods, systems, hardware, components and elements known in the art.

The invention has been described in detail with particular reference to example embodiments, but it will be understood that variations and modifications can be affected that are within the scope of the invention. For example, the operator could enter equivalent bounding box information and seed information in any of a plurality of ways, including pointing to a particular tooth or other object using a touch screen or making a text entry on a keyboard, for example. The presently disclosed example embodiments are, therefore, considered in all respects to be illustrative and not restrictive. The scope of the present invention is indicated by the appended claims, and all changes that come within the meaning and range of equivalents thereof are intended to be embraced therein.

What is claimed is:

1. A method for tooth segmentation, comprising:
acquiring a volume image of a patient;
identifying a first plane extending through maxillary portions of the patient's jaw and a second plane extending through mandibular portions of the patient's jaw; and
generating a maxillary sub-volume from the volume of interest according to the first plane and a mandibular sub-volume from the volume of interest according to the second plane.

2. The method of claim 1, wherein the method further comprises:
forming, for each sub-volume, a maximum intensity projection image MIP from voxels of the corresponding sub-volume;
delineating teeth from the MIP data to define tooth contour within each corresponding sub-volume; and
segmenting and displaying teeth within each respective sub-volume according to the tooth delineation.

3. The method of claim 1, wherein the step of identifying the first or second plane comprises accepting operator input for positioning the plane with respect to the volume image.

4. The method of claim 1, wherein the method further comprises a step of extracting a volume of interest comprising patient dentition from the acquired volume image.

5. The method of claim 1, wherein the step of identifying the first or second plane comprises processing volume data to align the plane to tooth structure.

6. The method of claim 1, wherein the step of acquiring the volume image comprises acquiring cone-beam computed tomography image content.

7. The method of claim 2, wherein the step of delineating teeth from the MIP data comprises a step of forming a spline corresponding to the arrangement of teeth in the sub-volume, and a step of calculating distances to tooth boundaries for points along the spline.

8. The method of claim 2, wherein the step of segmenting teeth comprises using a level set method.

9. The method of claim 2, wherein the step of forming the MIP for the maxillary or mandibular sub-volume comprises a step of defining and using a normal to the corresponding first or second plane.

10. The method of claim 2, wherein the method further comprises a step of executing a random walk algorithm on the MIP data.

11. The method of claim 2, wherein the method further comprises a step of computing a medial axis for one or more teeth.

12. A method for tooth segmentation, the method comprising the steps of:
acquiring a cone beam computed tomography volume image of a subject;
accepting an operator instruction that defines a first plane extending through maxillary portions of the patient's jaw and a second plane extending through mandibular portions of the patient's jaw;
generating a maxillary sub-volume from the volume of interest according to the first plane; and
generating a mandibular sub-volume from the volume of interest according to the second plane.

13. The method of claim 1, wherein the method further comprises the steps of:
generating, for each sub-volume, a 2D maximum intensity projection image from voxels of the corresponding sub-volume;
delineating teeth from the 2D MIP data within each corresponding sub-volume;
segmenting teeth within each respective sub-volume according to the tooth delineation; and
computing and displaying cephalometric parameters for diagnosis using the tooth segmentation.

14. The method of claim 12, wherein the method further comprises a step of extracting a volume of interest from the acquired volume image, wherein the volume of interest comprises patient dentition.

15. The method of claim 12, wherein the step of forming the mandibular sub-volume comprises the steps of using the portion of the volume image on one side of the second plane, and adding connected portions of the volume image that lie between the first and second planes.

16. The method of claim 13, wherein the step of generating the 2D maximum intensity projection image comprises assessing voxel values aligned along a normal to the first or second plane.

17. The method of claim 13, wherein the step of delineating teeth from the 2D MIP data further comprises applying a random walk algorithm.

18. The method of claim 13, wherein the step of computing and displaying cephalometric parameters comprises a step of displaying a medial axis for one or more segmented teeth.

19. The method of claim 13, wherein the step of segmenting further comprises a step of identifying one or more false negative or false positive conditions.

20. The method of claim 19, wherein the method further comprises the steps of correcting for the false positive condition by generating an intermediate result using a region-driven level-set segmentation, and processing the generated intermediate result by applying an edge-driven level set segmentation.

21. The method of claim 19, wherein the method further comprises identifying a region within a slice having a level-set transition from another slice and applying erosion over the identified region.

22. The method of claim 13, wherein the step of segmenting further comprises applying a shrink or expand force to a level-set segmentation algorithm.

23. An imaging apparatus, comprising:
an x-ray source and receiver configured to acquire a plurality of projection images of a patient;
a processor configured to:
(i) form a volume image of patient dentition from the acquired projection images;
(ii) identify a first plane extending through maxillary portions of the patient's jaw and a second plane extending through mandibular portions of the patient's jaw according to operator instructions;
(iii) generate a maxillary sub-volume from the volume of interest according to the first plane and a mandibular sub-volume from the volume of interest according to the second plane;
(iv) form, for each sub-volume, a maximum intensity projection image MIP from voxels of the corresponding sub-volume;
(v) delineate teeth from the MIP data to define tooth contour within each corresponding sub-volume; and
(vi) segment and display teeth within each respective sub-volume according to the tooth delineation.

24. The apparatus of claim 23, wherein the x-ray source and receiver are part of a cone beam computed tomography system.
